(19) United States
(12) Patent Application Publication
(10) Pub. No.: US 2007/0065456 A1
(43) Pub. Date: Mar. 22, 2007

(54) Nutritional supplements

(76) Inventor: Cindy J.P. Woods, San Francisco, CA (US)

Correspondence Address: FISH & RICHARDSON P.C., PO BOX 1022, MINNEAPOLIS, MN, 55440-1022, US

(21) Appl. No.: 11/522,830

(22) Filed: Sep. 18, 2006

Related U.S. Application Data
(60) Provisional application No. 60/718,888, filed on Sep. 20, 2005.

(51) Int. Cl. A61K 38/48; A61K 36/8962; A61K 36/55; A61K 36/28; A61K 31/045; A61K 33/04; A61K 33/32; A61K 31/205; A61K 31/385

(52) U.S. Cl. 424/195.17; 424/725; 424/764; 424/738; 514/2; 514/54; 514/440; 514/554; 514/562; 514/729; 424/641; 424/702; 424/639; 514/356; 514/688; 424/761; 424/735; 424/745; 424/754; 424/750; 424/768; 514/250; 424/756; 514/276; 514/393; 424/705; 514/563

(57) ABSTRACT

Nutritional supplement formulations are disclosed, such as nutritional supplement formulations that include Wasabia japonica. Methods for using the same to supplement a human diet are also disclosed.

### CROSS-REFERENCES TO RELATED APPLICATIONS

This application claims the benefit under 35 U.S.C. §119(e) of U.S. Provisional Application No. 60/718,888, filed Sep. 20, 2005, which is incorporated by reference in its entirety herein.

### TECHNICAL FIELD

This invention relates to nutritional supplements, and more particularly to nutritional supplements and methods for supplementing a human diet.

### BACKGROUND

Many people, including athletes, accumulate various toxins and heavy metals in their bodies due to dietary or environmental exposure. In response, some people attempt to improve their general health, well-being, and/or athletic performance through the inclusion of nutritional supplements in their diet. It would be useful to have nutritional supplements that could, for example, assist in the detoxification of a person's liver, blood, and/or intestinal tract.

Reduction of foreign substances, including heavy metals and environmental toxins, from the body could also have implications in the reduction of inflammation. Inflammation is a natural response to the presence of foreign substances or injury to the body, and is linked to the abnormal production of arachadonic acid. Removal of inflammatory triggers from the body can decrease the production of arachadonic acid and therefore reduce the manufacture of inflammatory messengers, including leukotrienes and prostaglandins, which are components of an inflammatory response. Diseases associated with an elevated level of inflammatory messengers which may be responsive to a decrease in their production include inflammatory bowel disease, allergies, asthma, gout, scleroderma, heart disease, rheumatoid arthritis, diabetes, Crohn's disease, and general aging.

### SUMMARY

This disclosure provides materials and methods for supplementing a human diet. For example, this disclosure provides nutritional supplement formulations. A nutritional supplement formulation can include Wasabia, such as Wasabia japonica. A nutritional supplement formulation can include EDTA, such as a time-release formulation of EDTA. The disclosure also provides materials such as kits that include one or more nutritional supplement formulations, such as nutritional supplement formulations that include Wasabia; nutritional supplement formulations that include EDTA, including time-release formulations of EDTA; nutritional supplement formulations that include dietary fiber sources, such as psyllium, celery powder, flax seed powder, and/or prune concentrate; and nutritional supplement formulations that include sources of sulfur or sulfur-containing compounds. Methods for supplementing a human diet by including one or more of the nutritional supplement formulations are also provided.

The nutritional supplement formulations, kits, and methods herein can be useful for improving a human's health and/or athletic performance. For example, an athlete can improve his or her athletic performance upon use of the formulations and methods disclosed herein. Use of the nutritional formulations can result in a decreased level of toxins, such as heavy metals (including mercury), aflatoxins, free radicals, fats, pollutants, and toxins in a human's blood, liver, and/or gastrointestinal tract. Reduction of these toxins can also yield a decline in the production of arachadonic acid, and/or inflammatory messengers such as leukotrienes and prostaglandins. These nutritional supplement formulations, kits and methods may therefore find use in the treatment of diseases responsive to a reduction in the inflammatory response, for example rheumatoid arthritis, lupus, allergies, inflammatory bowel disease, Chrohn's disease, ulcerative colitis, obesity, asthma, gout, scleroderma, psoriasis, heart disease, diabetes, and general aging.

In addition, the nutritional supplement formulations disclosed herein may also find use in modifying body composition, with optionally moderate changes in diet and exercise. Use of the nutritional formulations and methods disclosed herein could result in a decrease in one or more of body fat percentage, fat mass, and body weight, and/or in an increase in resting metabolic rate, fat free mass, and body weight. It is well understood that the average population is becoming increasing overweight and inactive, therefore a nutritional formulation that provides the results of dieting and exercise without much additional effort is desirable.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 summarizes four of the major categories of body composition monitored during the course of a four-week detoxification program. These categories include body fat, fat mass, lean mass, and weight. All values are shown in pounds.

FIG. 2 illustrates the average change in weight observed in 52 subjects following a four-week detoxification program involving the nutritional-supplements disclosed herein.

FIG. 3 depicts the average increase in weight in pounds observed following a four-week detoxification program.

### DETAILED DESCRIPTION

Provided herein are nutritional supplement formulations and methods for supplementing a human diet. The methods can include ingesting one or more of the described formulations for certain time periods and/or in a certain order. Kits comprising one or more of the nutritional supplement formulations are also provided.

Formulations

A number of nutritional supplement formulations are provided herein. A nutritional supplement formulation can be in any form, e.g., liquid, solid, gel, emulsion, powder, tablet, capsule, or gel cap (e.g., soft or hard gel cap). A nutritional supplement formulation typically will include one or more compositions that have been purified, isolated, or extracted (e.g., from plants) or synthesized, which are combined to provide a benefit (e.g., a health benefit in addition to a nutritional benefit) when used to supplement food in a diet.

In certain embodiments, recommended amounts per day or per serving of a nutritional supplement formulation or of ingredients provided in a nutritional supplement formulation are set forth herein. In certain cases, as will be recognized by one having ordinary skill in the art, one could vary the form of the nutritional supplement formulation, e.g., by substituting a powder for a capsule, a tablet for a capsule, a gel-cap for a tablet, a gel-cap for a capsule, a powder for a gel-cap, or any such combination, in order to provide such recommended amounts per day or per serving of a nutritional supplement formulation.

Any of the nutritional supplement formulations can be prepared using well known methods by those having ordinary skill in the art, e.g., by mixing the recited ingredients in the proper amounts. Ingredients for inclusion in a nutritional supplement formulation are generally commercially available, e.g., from Douglas Labs, Inc., B&D Nutritional Ingredients, Stella Labs, Nutricap Labs, Sedona Labs, Pharmline Inc., and Garden State Nutritionals.

One nutritional supplement formulation described herein includes one or more of psyllium powder, celery powder, prune concentrate, and flax seed powder. Such fiber sources can provide healthful benefits to the GI tract. In certain cases, such a nutritional supplement formulation can also comprise one or more of papain, betaine, pepsin, L-glutamine, fructooligosaccharides (FOS), and methylsulfonylmethane (MSM). MSM is thought to be effective to reduce muscle and joint pain. FOS is believed to prevent or ameliorate digestive ailments such as constipation and diarrhea, particularly in those who have finished a course of antibiotics, have visited a foreign country, or have a poor diet. One or more of bentonite powder, aloe vera powder, mint, lactobacillus acidophilus, anise, Vitamin C, bromelain, and magnesium can also optionally be included in such a nutritional supplement formulation.

One example of such a nutritional supplement formulation is the Ever Clean Brand Nutritional Supplement Formulation, which, when ingested as provided according to a method described herein, provides the following amounts of the following ingredients, among other ingredients, per serving:

| | |
|---|---|
| Betaine | 650 mg |
| Pepsin | 100 mg |
| L-glutamine | 500 mg |
| Fructooligosaccharides (FOS) | 2000 mg |
| Methylsulfonylmethane (MSM) | 1000 mg |

In some embodiments, a serving is about 2 tablespoons. In some embodiments, about 4 tablespoons per day are recommended. While not being bound by theory, it is believed that Ever Clean Powder can bind and immobilize toxic compounds such as aflatoxins in the gastrointestinal tract. The dietary fiber provided by psyllium powder, celery powder, prune concentrate, and flax seed powder also provides nutritional benefits.

Another nutritional supplement formulation provided herein can also include one or more of psyllium powder, celery powder, prune concentrate, and flax seed powder, and can further comprise one or more of garlic, barley, and chlorella, and optionally one or more of bentonite powder, aloe vera powder, mint, lactobacillus acidophilus, anise, Vitamin C, bromelain, pepain, and magnesium. While not being bound by theory, it is believed that the such a formulation is probiotic and detoxifying, helping to clean the gastrointestinal tract. For example, garlic is thought to boost antioxidant levels in the body and to reduce cholesterol levels, as well as to delay the growth of certain cancers. As described herein, garlic can be standardized to include 2000 ppm allicin.

One example of such a nutritional supplement formulation is available from Douglas Labs, Rocklin Calif., as 7594 DL Ultra D-Tox, which includes the following ingredients:

Bentonite Powder

Psyllium Powder

Lactobacillus Acidophilus (min. 1 billion CFU)

Garlic

Celery Powder

Aloe Vera Powder

Prune Concentrate

Mint

Flax Seed Powder

Barley

Chlorella

Anise

Vitamin C

Bromelain

Magnesium (citrate)

As used herein, such a formulation is also referred to as Meta Flush Brand Nutritional Supplement Formulation. Meta Flush can be provided in a capsule form. In some embodiments, a recommended dosage of a Meta Flush Capsule Formulation is three capsules (e.g., 1 in the morning, 2 in the evening).

Another nutritional supplement formulation described herein can include Wasabia japonica. Wasabia japonica (powder) is commercially available from B&D Nutritional Ingredients, Inc., and is prepared by harvesting Wasabia japonica plants, which are grown hydroponically, at maturity, separating the rhizome from the rest of the plant, and flash freezing the rhizome, to result in a powder. The powder can deliver at least 1500 μg isothiocyanates per gram of powder. In some cases, the powder is standardized to include 1500 μg of isothiocyanates per gram of powder. The isothiocyanates can include long chain methyl ITCs. The isothiocyanates can include one or more of allyl ITC, 3-butentyl ITC, and 4-pentenyl ITC.

Such nutritional supplement formulations comprising Wasabia japonica can further comprise Silybum marianum and/or Cynara scolymus, and optionally one or more of the following: choline bitartrate, L-methionine, inositol, betaine HCl, lecithin, niacin, taraxacum, curcumin, alpha lipoic acid, folic acid, manganese, selenium, taurine, zinc, phyllanthus (standardized to 0.02% phyllanthin and hypophyllanthin), picrorhiza (standardized to 4% kutkin), and trimethylglycine (TMG). While not limited by theory, it is believed that the such a formulation can protect the liver against various toxic stresses. Lipoic acid is a vitamin like substance that plays a key role in energy production and is thought to help stabilize glucose levels. Choline, betaine, and methionine are involved in methyl group metabolism, which is essential for normal liver function. Choline is used for synthesis of cell membranes. Taraxacum (dandelion) has been traditionally thought to detoxify the liver, while Silybum marianum (milk thistle) has also been shown to support healthy liver function under stress factors such as alcohol consumption.

Recommended dosage ranges for ingredients, when included, in such a nutritional supplement formulation range as described below (total amounts per day):

| | |
|---|---|
| Choline bitartrate | 750 mg-2500 mg |
| L-methionine | 5 mg-30 mg |
| Inositol | 50 mg-250 mg |
| Betaine HCl | 25 mg-150 mg |
| Lecithin | 25 mg-150 mg |
| Niacin | 2 mg-20 mg |
| Taraxacum | 10 mg-150 mg |
| Curcumin | 10 mg-150 mg |
| Silybum marianum | 10 mg-150 mg |
| Cynara | 10 mg-150 mg |
| Lipoic acid | 20 mg-150 mg |
| Folic acid | 50 μg-500 μg |
| Manganese | 1 mg-5 mg |
| Selenium | 25 μg-75 μg |
| Taurine | 2-8 mg |
| Zinc | 2 mg-10 mg |
| Phyllanthus | 150 mg-2000 mg |
| Picrorhiza (kurroa) | 200 mg-800 mg |
| Wasabia japonica | 100 mg-2000 mg |
| Trimethylglycine | 150 mg-1500 mg |

One example of such a nutritional supplement formulation is a Liver Clear Brand Nutritional Supplement formulation, wherein the recommended amount provided of each ingredient per day is as follows:

| | | |
|---|---|---|
| Choline bitartrate | 1500 | mg |
| L-methionine | 15 | mg |
| Inositol | 150 | mg |
| Betaine HCl | 75 | mg |
| Lecithin | 75 | mg |
| Niacin | 10 | mg |
| Taraxacum | 50 | mg |
| Curcumin | 50 | mg |
| Silybum marianum | 50 | mg |
| Cynara | 50 | mg |
| Lipoic acid | 100 | mg |
| Folic acid | 200 | μg |
| Manganese | 3 | mg |
| Selenium | 50 | μg |
| Taurine | 5 | mg |
| Zinc | 6 | mg |
| Phyllanthus | 1000 | mg |
| Picrorhiza (kurroa) | 500 | mg |
| Wasabia japonica | 400 | mg |
| Trimethylglycine | 750 | mg |

Liver Clear can be provided in tablet form, in certain embodiments.

Another nutritional supplement formulation described herein can include Silybum marianum and/or Cynara scolymus, and optionally one or more of the following: choline bitartrate, L-methionine, inositol, betaine HCl, lecithin, niacin, taraxacum, curcumin, alpha lipoic acid, folic acid, manganese, selenium, taurine, zinc, phyllanthus, picrorhiza, and trimethylglycine (TMG). The dosages of such ingredients can be as described above for nutritional supplements containing Wasabia japonica. One example of such a nutritional supplement formulation is the Liver Clear Brand Alternative Formulation, wherein the recommended dose per day is:

| | | |
|---|---|---|
| Choline bitartrate | 2000 | mg |
| L-methionine | 20 | mg |
| Inositol | 200 | mg |
| Betaine HCl | 100 | mg |
| Lecithin | 100 | mg |
| Niacin | 13.35 | mg |
| Taraxacum | 66.7 | mg |
| Curcumin | 66.7 | mg |
| Silybum marianum | 66.7 | mg |
| Cynara | 66.7 | mg |

Yet another nutritional supplement formulation described herein can include one or more sulfur-containing compounds, such as those selected from the group consisting of L-glutathione, N-acetyl-cysteine, dietary sulfur, selenomethionine, biotin, thiamine, and riboflavin. Sulfur-containing compounds include some strong antioxidants and some compounds that are thought to assist in maintaining joint health. For example, L-glutathione is a powerful antioxidant, and assists in the breakdown of toxins in the liver. Such formulations can further comprise one or more of garlic (powder, also a sulfur-containing compound), parsley (dried), tumeric, neem (standardized to 4% bitter principles), and triphala. It is believed that such formulations help support metabolic processes that protect against the toxicity of heavy metals, including mercury.

Dosage ranges for ingredients, when included, in such a nutritional supplement formulation range as described below (total amounts per day):

| | |
|---|---|
| L-glutathione (reduced) | 20 mg-100 mg |
| N-acetyl-L-cysteine | 100 mg-1000 mg |
| Dietary sulfur | 100 mg-1000 mg |
| Selenomethionine | 50 μg-150 μg |
| Biotin | 100 μg-400 μg |
| Thiamine | 10 mg-50 mg |
| Riboflavin | 5 mg-50 mg |
| Garlic (Pure-Gar) | 100 mg-500 mg |
| Parsley | 50 mg-150 mg |
| Tumeric | 400 mg-1500 mg |
| Neem | 200 mg-1000 mg |
| Triphala | 200 mg-1000 mg |

One example of such a nutritional supplement formulation is the Pure Plasma Brand Nutritional Supplement Formulation, wherein the recommended amount per day of each ingredient is:

| | | |
|---|---|---|
| L-glutathione (reduced) | 50 | mg |
| N-acetyl-L-cysteine | 500 | mg |
| Dietary sulfur | 500 | mg |
| Selenomethionine | 100 | μg |
| Biotin | 200 | μg |
| Thiamine | 25 | mg |
| Riboflavin | 10 | mg |
| Garlic (Pure-Gar) | 250 | mg |
| Parsley | 100 | mg |
| Tumeric | 750 | mg |
| Neem | 500 | mg |
| Triphala | 500 | mg |

Pure Plasma can be provided in capsule form in certain embodiments. In some embodiments, Pure Plasma can have the following alternative formulation (recommended amount per day):

Pure Plasma Capsules, alternative formulation

| | | |
|---|---|---|
| L-glutathione (reduced) | 50 | mg |
| N-acetyl-L-cysteine | 500 | mg |
| Dietary sulfur | 500 | mg |
| Selenomethionine | 100 | μg |
| Biotin | 200 | μg |
| Thiamine | 25 | mg |
| Riboflavin | 10 | mg |
| Garlic (Pure-Gar) | 250 | mg |
| Parsley | 100 | mg |

Nutritional supplement formulations comprising EDTA are also provided herein. EDTA can be provided in a total amount of about 500 mg to about 2500 mg per day. In some embodiments, EDTA is provided at a dosage of about 2000 mg/day, for example, in the form of capsules or gel-caps. Nutritional supplement formulations comprising a time-release EDTA formulation are also contemplated. Time-release formulation technology is well known to those having ordinary skill in the art; see, e.g., U.S. Pat. Nos. 6,191,162; 6,197,340; and 6,572,888.

Nutritional supplement formulations comprising eicosapentanoic acid (EPA) and/or gamma-linolenic acid (GLA) are also provided. In one embodiment, nutritional supplement formulations that include EPA can provide about 3 g/day of EPA, and/or about 3 g/day of GLA. EPA and GLA can be in the form of soft-gel caps, in some embodiments.

In a further embodiment, a nutritional supplement formulation may combine the ingredients from two or more of the above formulations. For example, a nutritional supplement formulation may contain ingredients consistent with a combination of Ever Clean Brand Nutritional Supplement Formulation and Meta Flush Brand Nutritional Supplement Formulation. One example of such a nutritional supplement formulation, when ingested as provided according to a method described herein, provides the following amounts of the flowing ingredients, among other ingredients, per serving:

| | | |
|---|---|---|
| Betaine | 650 | mg |
| Pepsin | 100 | mg |
| L-glutamine | 500 | mg |
| Fructooligosaccharides (FOS) | 2000 | mg |
| Methylsulfonylmethane (MSM) | 1000 | mg |
| Proprietary Blend | 18.6 | g |

where the Proprietary Blend includes: Bentonite Powder, Psyllium Powder (husks), Celery Powder (seed), Aloe Vera Powder (leaf), Prune Concentrate (fruit), Mint (leaf), Lactobacillus Acidophilus, Papain, Flax Powder (seed), Anise (seed), Vitamin C, Bromelain, and Magnesium.

In another embodiment, a nutritional supplement formulation may contain ingredients consistent with a combination of Liver Clear Brand Nutritional Supplement Formulation and Pure Plasma Brand Nutritional Supplement Formulation. One example of such a nutritional supplement contains the following recommended amount per day of each ingredient:

| | | |
|---|---|---|
| Choline bitartrate | 1500 | mg |
| L-methionine | 15 | mg |
| Inositol | 150 | mg |
| Betaine HCl | 75 | mg |
| Lecithin | 75 | mg |
| Niacin | 10 | mg |
| Taraxacum | 50 | mg |
| Tumeric (Curcumin) | 800 | mg |
| Silybum marianum | 50 | mg |
| Cynara | 50 | mg |
| Lipoic acid | 100 | mg |
| Folic acid | 200 | μg |
| Manganese | 3 | mg |
| Selenium | 150 | μg |
| Taurine | 5 | mg |
| Zinc | 6 | mg |
| Phyllanthus | 1000 | mg |
| Picrorhiza (kurroa) | 500 | mg |
| Wasabia japonica | 400 | mg |
| Trimethylglycine | 750 | mg. |
| L-glutathione (reduced) | 50 | mg |
| N-acetyl-L-cysteine | 500 | mg |
| Dietary sulfur | 500 | mg |
| Biotin | 200 | μg |
| Thiamine | 25 | mg |
| Riboflavin | 10 | mg |
| Garlic (Pure-Gar) | 250 | mg |
| Parsley | 100 | mg |
| Neem | 500 | mg |
| Triphala | 500 | mg |

In a further embodiment, a nutritional supplement formulation may contain ingredients consistent with the combination of Meta Flush Brand Nutritional Supplement Formulation, Liver Clear Brand Nutritional Supplement Formulation and Pure Plasma Brand Nutritional Supplement Formulation. One example of such a nutritional supplement formulation contains the following dosages of ingredients (total amounts per serving):

| | | |
|---|---|---|
| Vitamin C | 90 | mg |
| Choline bitartrate | 1500 | mg |
| L-methionine | 15 | mg |
| Inositol | 150 | mg |
| Betaine HCl | 75 | mg |
| Lecithin | 75 | mg |
| Niacin | 10 | mg |
| Taraxacum | 50 | mg |
| Turmeric (Curcumin) | 800 | mg |
| Silybum marianum | 50 | mg |
| Cynara | 50 | mg |
| Lipoic acid | 100 | mg |
| Folic acid | 200 | μg |
| Manganese | 3 | mg |
| Selenium | 150 | μg |
| Taurine | 5 | mg |
| Zinc | 6 | mg |
| Phyllanthus | 1000 | mg |
| Picrorhiza (kurroa) | 500 | mg |
| Wasabia japonica | 400 | mg |
| Trimethylglycine | 750 | mg. |
| L-glutathione (reduced) | 50 | mg |
| N-acetyl-L-cysteine | 500 | mg |
| Dietary sulfur | 500 | mg |
| Biotin | 200 | μg |
| Thiamine | 25 | mg |
| Riboflavin | 10 | mg |
| Garlic (Pure-Gar) | 250 | mg |
| Parsley | 100 | mg |
| Neem | 500 | mg |
| Triphala | 500 | mg |
| Proprietary Blend | 1,410 | mg |

where the Proprietary Blend includes: Bentonite Powder, Psyllium Powder (husks), Celery Powder (seed), Aloe Vera Powder (leaf), Prune Concentrate (fruit), Mint (leaf), Lactobacillus Acidophilus, Flax Powder (seed), Anise (seed), Bromelain, and Magnesium.

Any of the nutritional supplement formulations provided herein can be provided in a variety of forms, e.g., liquid, gel, solid, emulsion, pill, caplet, tablet, capsule, powder, hard cap, gel cap, etc., and packaged in a variety of containers, canisters, blister paks, etc., as well known to those having ordinary skill in the art. For oral administration, soft gel capsules can be prepared by conventional means with pharmaceutically acceptable excipients such as binding agents, fillers, lubricants, disintegrants, or wetting agents. Soft gel capsule manufacturing methods are described in, e.g., U.S. Pat. No. 6,333,047.

Kits

Kits including one or more of the nutritional supplements described previously are also provided. For example, a kit can include a nutritional supplement comprising Wasabia japonica. A kit can include a nutritional supplement formulation comprising EDTA. A kit can include a nutritional supplement formulation that includes EPA and/or GLA. Optional ingredients in the kits include measuring spoons or cups; spoons; straws; and instructions for methods of use.

Methods of Use

Any of the described nutritional supplement formulations can be used in a method to supplement a human diet. Also provided herein are methods to enhance athletic performance, to reduce weight, to increase weight, to detoxify the blood, liver, and/or GI tract, to modify body composition, and to decrease inflammation. Further provided herein are methods to reduce the level of C-reactive protein, inflammatory messenger precursors, and/or heavy metals in a human. Any of the methods described herein include ingestion of one or more nutritional supplement formulations, e.g., by a human. The methods disclosed herein may include administration of one or more nutritional supplement formulations. A nutritional supplement formulation can be ingested alone or in combination with any other nutritional supplement formulation, in any order and for varying relative lengths of time. In certain cases, certain formulations are used prior to other formulations, while other formulations are ingested concurrently.

In some embodiments, Ever Clean Powder as described above can be used in a method to supplement a diet. In certain cases, Ever Clean Powder can be ingested in an amount of about 0.5 tablespoons to about 5 tablespoons. Ever Clean Powder can be ingested once a day, or twice a day, e.g., in the morning and at night. Ever Clean Powder can be used for a period of from about 1 day to about 18 days, such as from about 5 days to about 15 days, or from about 7 days to about 14 days (e.g., 8, 9, 10, 11, 12, or 13 days).

In certain embodiments, Ever Clean Powder can be used in a method to supplement a diet in combination with other nutritional supplement formulations described herein. For example, in one four week protocol, Ever Clean Powder can be ingested as described for approximately 10 to 15 days, such as for about 12 days. A second nutritional supplement formulation can be ingested on the same days as the Ever Clean Powder (e.g., an EDTA nutritional supplement formulation, an EPA and/or GLA nutritional supplement formulation, or a Liver Clear Tablet formulation (all as described above)). These additional nutritional supplement formulations can also be ingested prior to or subsequently to completion of a course of ingestion of Ever Clean Powder, e.g., for one or more days prior to, or after completion of, one or more days of ingestion of Ever Clean Powder. For example, an EDTA supplement formulation can be ingested during the course of ingestion of Ever Clean Powder and continued thereafter for about 1 day to about 18 days (e.g., about 5, 10, 12, 14, 15, 16, 17, or 18 days) after completion of one or more days of ingestion of Ever Clean Powder. Similarly, a Liver Clear Tablet and/or EPA and/or GLA nutritional supplement formulation can be ingested during the course of Ever Clean Powder ingestion and continued thereafter for about 1 day to about 18 days (e.g., about 5, 10, 12, 14, 15, 16, 17, or 18 days) after completion of one or more days of ingestion of Ever Clean Powder.

In certain cases, a method of supplementing a diet includes ingesting one or more additional nutritional supplement formulations following completion of a certain number of days of ingestion of Ever Clean Powder. For example, a Pure Plasma Formulation, as described above, can be ingested for one or more days after completion of a number of days of ingestion of Ever Clean Powder. Thus, a Pure Plasma Formulation can be ingested after about 5-10 days (e.g., about 7 days) ingestion of Ever Clean Powder, and continue for a time period of about 1 day to about 25 days, e.g., about 7 days to about 21 days, or about 18 to about 21 days.

Another nutritional supplement formulation that can be ingested following a completion of a certain number of days of ingestion of Ever Clean Powder is the Meta Flush Capsule Formulation described above. For example, a Meta Flush Capsule Formulation can be ingested for one or more days after completion of about 7-17 days (e.g., about 10, 12, 14, 15, or 16 days) ingestion of Ever Clean Powder, and continue for a time period of about 1 day to about 20 days, e.g., about 7 days to about 14 days. In certain cases, a Meta Flush Capsule Formulation course of ingestion is commenced only after completion of a course of ingestion of Ever Clean Powder. For example, a Meta Flush Capsule Formulation course of ingestion can be commenced from about 1 day to about 5 days after completion of a course of ingestion of Ever Clean Powder, e.g., where the Ever Clean Powder course of ingestion lasted about 10 to about 15 days.

A method of reducing inflammation described herein can include ingestion of one or more nutritional supplement formulations, e.g., by a human. Characteristics of inflammation include one or more of the following: redness; swollen joints, which may be warm to the touch; joint pain; joint stiffness; or loss of joint function. Other symptoms may include fever, chills, fatigue, headaches, loss of appetite, and muscle stiffness. Inflammation can be chronic, acute, or both. Acute inflammation is described as the initial response of the body when living tissue is subject to injury. The response is generally rapid and complete within a time-frame of hours to days. When inflammation does not subside over the course of a few days, the inflammatory response has entered a chronic state. Chronic inflammation can occur when the stimulus to inflammation in the tissues is persistent; the duration of the response can be weeks, months, or even indefinitely. The lengthened process, in conjunction with the body's repeated attempts at healing and repair, inevitably leads to tissue damage. Many diseases are associated with chronic inflammation and can include: allergies, Alzheimer's, anemia, aortic valve stenosis, arthritis, cancer, congestive heart failure, fibromyalgia, fibrosis, heart attack, kidney failure, lupus, pancreatitis, psoriasis, stroke, and surgical complications.

Inflammation, as described herein, can be the result of foreign substances within the body or injury to the body. A foreign substance may include: heavy metals, e.g., mercury, aluminum, arsenic, cadmium, lead, and thallium; environmental air pollutants, e.g, pesticides or diesel exhaust; foods and food additives, e.g., pesticides, preservatives, waxing on fruits and vegetables, artificial colors, and ripening agents; water contaminants and additives; pharmaceuticals and other consumer products. The presence of these foreign substances within the body can begin a cascade of reactions which initiate an inflammatory response. This cascade is marked by the production of inflammatory messengers, e.g., leukotrienes and prostaglandins. Examples of the leukotrienes and prostaglandins involved in the inflammatory response include LTB₄ and PGE₂. These inflammatory messengers are produced in response to elevated levels of the inflammatory messenger precursor AA. Inhibition of these inflammatory messengers can result from increased levels of the inflammatory messenger precursors EPA, docosahexaenoic acid (“DHA”), GLA, and dihomo-gamma-linolenic acid (“DGLA”). It is possible to quantitate the reduction in inflammation by monitoring the levels of these precursors and that of C-reactive protein (“CRP”) in the bloodstream. When inflammation is chronic and persistent, levels of CRP will be high. A diminution in the level of any member of the inflammatory cascade, e.g., CRP, AA, leukotrienes, or prostaglandins, can indicate a decline in the inflammatory response. While not limited by theory, it is believed that the nutritional formulations described herein are capable of decreasing the levels of foreign substances within the body (e.g., heavy metals), wherein a decline in said foreign substances as described herein may be linked to the reduction or prevention of inflammation. It is also believed that the reduction of an inflammatory response may be coupled to the reduction of the inflammatory messenger precursor AA and the resultant inflammatory messengers as initiated by the introduction of the nutritional formulations described herein.

In certain embodiments, the use of a nutritional supplement formulation, such as described herein, may result in one or more changes in overall body composition. While weight is an important consideration to overall health, some consider a better measure of health and fitness to be body composition. A method of modifying body composition as illustrated herein can include ingestion or administration of one or more nutritional supplement formulations, e.g., by a human. Modifications may include one or more of an increase in overall metabolic rate (e.g., calories needed during rest, activity and exercise), resting metabolic rate, fat free mass (i.e., muscle or lean mass), and body weight. Changes to body composition may include one or more of a decrease in body fat percentage, fat mass, and body weight. Measurements of these characteristics of body composition are well understood by those of skill in the art. Modifications of body composition may be determined through the comparison of values obtained in a human prior to administration of a nutritional supplement formulation to those obtained during or at the completion of the method. In some embodiments, the method may be modified as instructed by a physician. A method as described using the nutritional supplement formulations and kits herein may offer a route to changes in body composition without the addition of restrictive dieting and exercise. In another embodiment, the nutritional supplements contained herein may also be administered alone or in varying combinations and dosages.

Links between metabolism and a reduction in the inflammatory response have been well documented (see, e.g., Wellen, K. E. et al., The Journal of Clinical Investigation 2005, 115(5), 1111; Maeda, K. et al., Cell Metabolism 2005, 1, 107; Knobler, H. et al., Q J Med 1999, 92, 73; and Makowski, L. and Hotamisligil, G. S., Nutrition and Gene Regulation, 2004, 2465S). These and other studies have found that obesity can be correlated with increased occurrences of inflammatory disorders such as diabetes, fatty liver disease, airway inflammation, and atherosclerosis. As one example, the liver plays a central role in the processing of all which is ingested in the body. Hepatitis, as a disease, is the result of toxic inflammation of the liver. There is ample evidence to suggest that toxins in the body, either from environmental factors or from natural ones (e.g., cortisol, free radicals, etc.) may set off an immune system inflammatory response. Furthermore, the links between the fat pathway and toxins, inflammation, and obesity have been well established. Toxins that cause inflammation may exacerbate the ability to lose weight or impact the insulin response that contributes to diabetes and obesity.

In one embodiment of the present disclosure, the causes of inflammation may be removed through the implementation of a method as described herein. In other embodiments, the administration of any or all of the nutritional supplements disclosed herein may also result in a decrease in inflammation and body weight. A reduction of weight and body fat may indicate a reduction in toxins within the body. This reduction may also be directly linked to reductions in the inflammatory response observed in a subject. Moreover, supplementing a diet using the nutritional supplements and methods described herein may allow for toxins to be eliminated from many areas of the body simultaneously. This prevents the adverse effects of only cleansing a single part of the body. For example, if one clears the colon but not the liver, inflammatory toxins remaining in the body are left to re-circulate in the blood and other organs.

### EXAMPLES

### Example 1

Nutritional Supplement Formulations:

The following nutritional supplement formulations were prepared by combining the listed ingredients in the appropriate amounts:

A. Liver Clear Tablets

4 tablets contain:

| | | |
|---|---|---|
| Choline bitartrate | 2000 | mg |
| L-methionine | 20 | mg |
| Inositol | 200 | mg |
| Betaine HCl | 100 | mg |
| Lecithin | 100 | mg |
| Niacin | 13.35 | mg |
| Taraxacum | 66.7 | mg |
| Curcumin | 66.7 | mg |
| Silybum marianum | 66.7 | mg |
| Cynara | 66.7 | mg |
| Lipoic acid | 133.4 | mg |
| Folic acid | 266.7 | μg |
| Manganese | 4 | mg |
| Selenium | 66.7 | μg |
| Taurine | 6.67 | g |
| Zinc | 8 | mg |
| Phyllanthus | 1333.33 | mg |
| Picrorhiza (kurroa) | 666.7 | mg |
| Wasabia japonica | 533.34 | mg |
| Trimethylglycine | 1000 | mg |

In some cases, Liver Clear can have the following alternative formulation, which does not include Wasabia japonica:

Liver Clear Tablets, Alternative Formulation

4 tablets contain:

| | |
|---|---|
| Choline bitartrate | 2000 mg |
| L-methionine | 20 mg |
| Inositol | 200 mg |
| Betaine HCl | 100 mg |
| Lecithin | 100 mg |
| Niacin | 13.35 mg |
| Taraxacum | 66.7 mg |
| Curcumin | 66.7 mg |
| Silybum marianum | 66.7 mg |
| Cynara | 66.7 mg |

B. Pure Plasma Capsules

2 capsules contain:

| | | |
|---|---|---|
| L-glutathione (reduced) | 50 | mg |
| N-acetyl-L-cysteine | 500 | mg |
| Dietary sulfur | 500 | mg |
| Selenomethionine | 100 | μg |
| Biotin | 200 | μg |
| Thiamine | 25 | mg |
| Riboflavin | 10 | mg |
| Garlic (Pure-Gar) | 250 | mg |
| Parsley | 100 | mg |
| Tumeric | 750 | mg |
| Neem | 500 | mg |
| Triphala | 500 | mg |

In some embodiments, Pure Plasma capsules can have the following formulation:

Pure Plasma Capsules, Alternative Formulation

2 capsules contain:

| | | |
|---|---|---|
| L-glutathione (reduced) | 50 | mg |
| N-acetyl-L-cysteine | 500 | mg |
| Dietary sulfur | 500 | mg |
| Selenomethionine | 100 | μg |
| Biotin | 200 | μg |
| Thiamine | 25 | mg |
| Riboflavin | 10 | mg |
| Garlic (Pure-Gar) | 250 | mg |
| Parsley | 100 | mg |

### Example 2

Methods for Supplementing a Diet

One method for supplementing a diet using the formulations described above can be as set forth below. The method involves ingesting the nutritional supplement formulations provided herein in a particular sequence. The method can also be used to enhance athletic performance, to reduce weight, to detoxify the blood, liver, and/or GI tract, and to reduce inflammation. The method is designed as an additive process that lasts for four weeks.

Week One

Ever Clean Powder

Dosage 2 tablespoons in the morning and 2 tablespoons at night

On day one, the person whose diet is to be supplemented begins ingestion of a course of Ever Clean cleansing powder. Typically, the person will begin in the evening to gauge how quickly his body will respond. If he does not have a bowel movement in the first 12 hours, the dose should be increased to 2 tablespoons 3 times a day. The person should continue for 12 days, or finish the entire jar of powder (4 tablespoons/day×12 days=48 tablespoons), whichever comes first.

Liver Clear Tablets Blister Pack

Dosage Liver Clear: 2 tablets in the morning and 2 at night

Also on day one, the person should begin using the Liver Clear blister packs. The person should begin on the same evening he ingests the first dosage of Ever Clean powder. After the first day, the person should take one blister pack (containing 2 tablets) in the morning and one (containing 2 tablets) in the evening, preferably with meals.

Week Two

Liver Clear & Pure Plasma Blister Packs

Dosage 1 Pure Plasma capsule in the morning, 1 at night

2 Liver Clear tablets in the morning, 2 at night

In week two, add the Pure Plasma capsules. Take one blister pack (containing 1 capsule) in the morning and one (containing 1 capsule) at night of Pure Plasma. Continue with the Liver Clear Tablet Blister Packs as in Week One. Continue to take Ever Clean as described above, e.g., up to day 12.

Weeks Three and Four

Meta Flush, Liver Clear, and Pure Plasma Blister Packs

Dosage 1 blister pack in the morning and 1 at night of each

In week three, add the Meta Flush blister packs (containing 1 capsule each) to the Liver Clear and Pure Plasma ingestion regimen. Continue taking all of the nutritional supplement formulations through week four.

Optional variation: In some embodiments, ingestion of an EDTA-containing nutritional supplement formulations can be included in the above-described method. For example, EDTA tablets, such as time-release EDTA tablets, having a total dosage of EDTA of from about 500 to about 2500 mg of EDTA, can be ingested during all or part of the four week regimen. In certain cases, EDTA tablets are ingested for the entire four week regimen, in a dosage of about 2000 mg EDTA/day.

In some embodiments, ingestion of EPA and/or GLA-containing nutritional supplement formulations can be included in the above-described method. For example, EPA capsules or gel-caps containing a total of about 3 g/day EPA and GLA capsules or gel-caps containing a total of about 3 g/day GLA can be ingested during all or part of the four week regimen. In certain cases, both EPA and GLA capsules or gel-caps are ingested for the entire four week regimen, in a dosage of about 3 g EPA per day and about 3 g GLA per day.

### Example 3

Effects of Detoxification on Organ Function, Heavy Metals Reduction, and Inflammatory Responses in the Body

A group of 50-100 individuals aged 30-50 undergo a four-week detoxification program involving the use of powders, capsules and tablets. Subjects are chosen to cover a cross-section of ethnicity and gender while also including those of average to obese weight with a variety of diets and exercise habits. Specific populations known to have high levels of AA in the bloodstream are incorporated into the study as well as those who suffer from diabetes, rheumatoid arthritis and those at risk for arthrosclerosis.

The dietary supplement regime used in this trial comprises Ever Clean Powder, Liver Clear Tablets Blister Pack, Pure Plasma Blister Packs, and Meta Flush with optional additional variants as described in the previous example. Participants are randomized into three groups. Subjects in the first group, the control group, receive no treatment. Those in the second group receive the described four-week protocol, while those in the third group receive the four-week protocol with the addition of EDTA, GLA and EPA.

A series of measurements are made throughout the study to quantify the changes observed as a result of the four-week detoxification program. Subjects undergo blood tests at the beginning of week 1, and at the conclusion of the study, the end of week four. These tests measure changes in the levels of AA, DHA, GLA, DGLA, CRP. The levels of heavy metals will also be tested. These tests include determining the levels of aluminum and mercury, but also test the levels of lead, cadmium, arsenic and thallium. Liver function tests, which include measures of the levels of alkaline phosphatase (“ALP”), alanine aminotransferase (“ALT”), aspartate aminotransferase (“AST”), bilirubin, gamma glutamic transpeptidase (“GGT”), albumin, and total protein, as well as complete blood counts, to quantitate levels of red blood cells, white blood cells, hemoglobin, and platelets, are also conducted. A resting metabolic rate measurement is made at the beginning and end of the study. Participants also complete a daily symptom checklist as a self-report measure to record both their physical and psychological symptoms and responses to the program. It is anticipated that the impact of detoxification on the system will cause an overall increase in the physiological functioning of the participants. This response is measured by a decrease in the levels of AA, CRP, and heavy metals in the blood in conjunction with improved liver function as indicated by levels of ALP, ALT, AST, bilirubin, GGT, albumin and total protein within an allowable range.

### Example 4

Effects of Detoxification on Weight, Fat Mass, Fat Free (Muscle) Mass, Body Weight, and Body Fat Percentage

52 men and women between the ages of 21 and 53 years old were the focus of a study to determine if subjects could see a change in weight, body fat percentage, fat free mass, fat mass, and resting metabolic rate over a four week period without special attention to diet or exercise. Subjects were drawn from patients of a clinical practice for nutrition and athletic performance over a three year period. Subjects included both professional athletes and average individuals with varying degrees of fitness.

Prior to beginning the method, a baseline measurement was taken using the Tanita 300, a professional body fat analyzer using bioimpedience methodology and measurement. The test provided information on each subject regarding their weight, body mass index (BMI), percent fat, fat mass (FM), fat free mass (FFM), total body weight (TBW) and an estimate of their resting metabolic rate (Kcal). Subjects were also tested with the KORR MetaCheck metabolic tester to determine their individual resting metabolic rate. Because food and exercise can increase the metabolic rate as the body either processes the food or increases the heart rate at exercise, each test was given in the morning prior to the subject eating, drinking, or exercising. Tests were repeated at the end of the four week period.

Each subject was administered an identical set of products that included:
One (1) Canister Ever Clean Powder
One (1) Canister Liver Clear Week One Supplement Packets
One (1) Canister of Liver Clear Pure Plasma Week Two Supplement Packets
One (1) Canister of Meta Flush Week Three and Four Supplement Packets
   Each subject underwent the same four week regimen as follows:
   Week One
   Products:
Ever Clean Powder
Liver Clear Supplement Packets
   Instructions:
Starting in the evening, subjects were instructed to mix 2 tablespoons of Ever Clean Powder with 6 ounces of water or juice, repeating the dosage three times daily for 12 days or until canister was empty, whichever comes first. Also on day one, the subject began using the Liver Clear blister packs. After the first day, the subject took one blister pack (containing 2 tablets) in the morning and one (containing 2 tablets) in the evening, preferably with meals.
   Week Two
   Products:
   Ever Clean Powder
   Liver Clear Pure Plasma Supplement Packets
      Instructions:
During week two, subjects completed the remaining Ever Clean Powder. The first morning of week two, subjects were instructed to begin taking the Liver Clear Pure Plasma Supplements. One pack was taken with the morning meal and one pack was taken with the evening meal.
   Week Three & Four
   Products:
   Meta Flush Supplement Packets
      Instructions:
Subjects began week three by taking the Meta Flush Supplements. During weeks three and four subjects took one pack with the morning meal and one pack with the evening meal.

No special diet requirements were mandated for the subjects, but subjects were instructed to refrain from alcohol and other recreational drugs. Additionally, subjects were asked to avoid caffeine, fumes (e.g., from paint and exhaust), herbal supplements (e.g., St. John's wort, garlic, and Schisandra berry), some medications (e.g., steroids and Phenobarbital and other barbiturates), nicotine, and pesticides as some foods and environmental items artificially speed the cleansing process.

The results of this study are summarized in Table 1, while each individual test and its results are discussed in detail below.

Increase in Resting Metabolic Rate (RMR)

Of the total subjects who participated, 4.43% of subjects saw an increase in metabolic rate or an increase of 98.48 calories burned. Of the total subject pool, 70.37% of subjects saw an increase in RMR. When considering only the subjects who saw an increase in their overall metabolic rate, they burned an average of 278.63 calories and saw an increase of 11.64% in RMR. On average, male subjects saw a total increase of 307.06 calories or 12.57% increase in RMR over their baseline, while female subjects saw a total increase of 127 calories burned and a 6.67% increase in their RMR.

Decrease in Body Fat Percentage

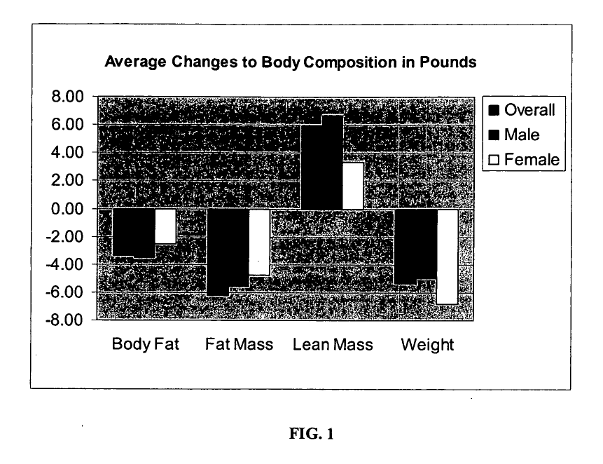

79.59% of subjects saw a decrease in their body fat percentage (FIG. 1). The average decrease in body fat per subject was 3.3 percentage points, or 19.89%. Of males who saw a decrease, each saw an average decrease of 3.5 percentage points, or 22.16%. Females who saw a decrease experienced an average reduction in body fat of 2.6 percentage points, or 9.83%.

Decrease in Fat Mass (FM)

In total, the subjects saw a decrease in body fat mass of 1.56 pounds, or 0.84% (FIG. 1). In males, the average per person decrease was 0.60 pounds, or 1.08%, while in females a decrease of 1.29% or 0.31 pounds was observed. Of the 63.16% of subjects who saw a decrease in fat mass, the average decrease in pounds per individual was 6.22 pounds of 14.48%. In males who saw a decrease in fat mass, the average total pounds lost was 5.57 pounds or 15.42%, in females the average individual lose was 4.73 pounds or 11.66%.

Increase of Fat Free MASS (FFM) (Muscle)

On average all subjects saw an increase in FFM (or lean mass) of 3.16 pounds or 2.02% (FIG. 1). A total of 71.05% of subjects saw an increase in FFM. Of those who saw an increase, the average increase in FFM was 5.92 pounds of 3.77%. By gender, males saw an individual average increase of 6.65 pounds of 3.90%, while females saw an average increase of 3.35 pounds or 3.33%

Decrease in Body Weight

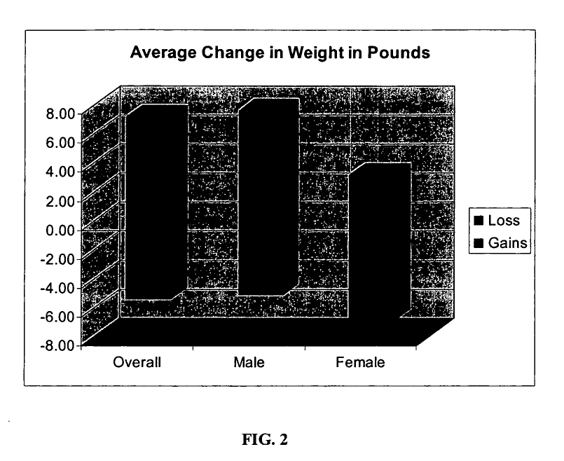

The percentage decrease in weight of the group as a whole was 0.90% or 1.43 pounds (FIG. 2). Men saw a slightly higher decrease in weight, 1.50 pounds or 0.87%, while women saw an average weight loss of 1.40 pounds or 1.06%. Of the total sample, 46.15% saw weight loss. Of those who experienced a loss in weight, the average decrease was 5.31 pounds or 2.51%. In males, the average weight loss equaled 5.01 pounds or 2.15%. In females the average total weight loss was 6.83 pounds of 4.33%.

Increase in Body Weight

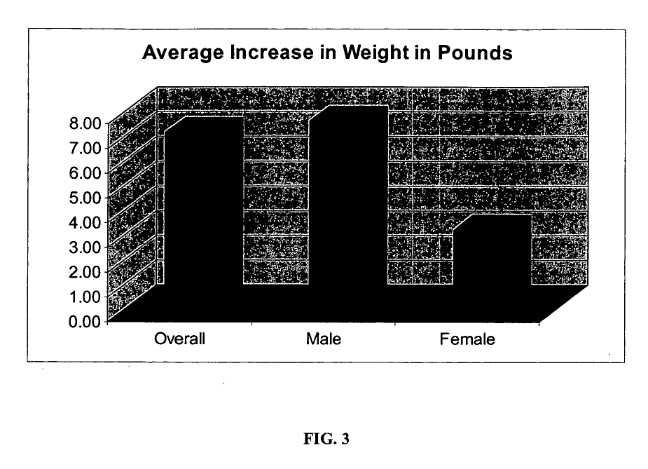

Of the total pool of subjects, 53.85% saw an increase in body weight (FIG. 3). The average increase in weight was 7.20 pounds of 3.81%. For males, the average pounds increased was 7.76 or 3.98%, and for females, the average increase was 3.27 pounds or 2.46%.

TABLE 1
Results.

| | Percent of | | | | | | | | | |
|---|---|---|---|---|---|---|---|---|---|---|
| | subjects | Average | | | Males | | | Females | | |
| | observing a | | | Percent | | | Percent | | | Percent |
| | change | Change | | Change | Change | | Change | Change | | Change |
| Decrease in | 79.59% | 3.33 | lbs | 19.89% | 3.50 | lbs | 22.16% | 2.57 | lbs | 9.83% |
| Body Fat | | | | | | | | | | |
| Decrease in | 63.16% | 6.22 | lbs | 14.48% | 5.57 | lbs | 15.42% | 4.73 | lbs | 11.66% |
| Fat Mass | | | | | | | | | | |
| Increase in | 71.05% | 5.92 | lbs | 3.77% | 6.65 | lbs | 3.90% | 3.35 | lbs | 3.33% |
| Lean Mass | | | | | | | | | | |
| Decrease in | 46.15% | 5.31 | lbs | 2.51% | 5.01 | lbs | 2.15% | 6.83 | lbs | 4.33% |
| Weight | | | | | | | | | | |
| Increase in | 53.85% | 7.20 | lbs | 3.81% | 7.67 | lbs | 3.98% | 3.27 | lbs | 2.46% |
| Weight | | | | | | | | | | |
| Increase in | 70.37% | 278.63 | kcal | 11.64% | 307.06 | kcal | 12.57% | 127.00 | kcal | 6.67% |
| RMR | | | | | | | | | | |

A number of embodiments of the invention have been described. Nevertheless, it will be understood that various modifications may be made without departing from the spirit and scope of the invention. Accordingly, other embodiments are within the scope of the following claims.

What is claimed is:

1. A method for supplementing a human diet, the method comprising:
a) ingesting a nutritional supplement formulation comprising Wasabia japonica.

2. The method of claim 1, wherein said nutritional supplement formulation further comprises Silybum marianum.

3. The method of claim 2, wherein said nutritional supplement formulation further comprises Cynara scolymus.

4. The method of claim 3, wherein said nutritional supplement formulation further comprises one or more of the following: choline bitartrate, L-methionine, inositol, betaine HCl, lecithin, niacin, taraxacum, curcumin, alpha lipoic acid, folic acid, manganese, selenium, taurine, zinc, phyllanthus, picrorhiza, and trimethylglycine (TMG).

5. The method of claim 1, wherein said method further comprises ingesting a second nutritional supplement formulation comprising EDTA.

6. The method of claim 5, wherein said second nutritional supplement formulation comprises a time-release formulation of EDTA.

7. The method of claim 5, wherein said method further comprises ingesting a third nutritional supplement formulation comprising one or more of psyllium powder, celery powder, prune concentrate, and flax seed powder.

8. The method of claim 7, wherein said third nutritional supplement formulation further comprises one or more of papain, betaine, pepsin, L-glutamine, fructooligosaccharides (FOS), and methylsulfonylmethane (MSM).

9. The method of claim 7, wherein said third nutritional supplement formulation further comprises one or more of garlic, barley, and chlorella.

10. The method of claim 8 or 9, wherein said third nutritional supplement formulation further comprises one or more of bentonite powder, aloe vera powder, mint, lactobacillus acidophilus, anise, Vitamin C, bromelain, and magnesium.

11. The method of claim 8 or 9, further comprising ingesting a fourth nutritional supplement formulation comprising one or more sulfur-containing compounds.

12. The method of claim 11, wherein said one or more sulfur-containing compounds is selected from the group consisting of L-glutathionine, N-acetyl-cysteine, dietary sulfur, selenomethionine, biotin, thiamine, and riboflavin.

13. The method of claim 11, wherein said fourth nutritional supplement formulation further comprises one or more of garlic, parsley, tumeric, neem, and triphala.

14. A method of supplementing a human diet, said method comprising:
a) ingesting a first nutritional supplement formulation comprising one or more of psyllium powder, celery powder, prune concentrate, and flax seed powder in combination with one or more of betaine, pepsin, L-glutamine, FOS, and MSM;
b) ingesting a second nutritional supplement formulation comprising Wasabia japonica;
c) ingesting a third nutritional supplement formulation comprising one or more sulfur-containing compounds; and
d) ingesting a fourth nutritional supplement formulation comprising one or more of psyllium powder, celery powder, prune concentrate, and flax seed powder in combination with one or more of garlic, barley, and chlorella.

15. The method of claim 14, further comprising ingesting a nutritional supplement formulation comprising EDTA.

16. The method of claim 14, wherein said first nutritional supplement formulation further comprises one or more of bentonite powder, aloe vera powder, mint, lactobacillus acidophilus, anise, Vitamin C, bromelain, and magnesium.

17. The method of claim 16, wherein said first nutritional supplement formulation is ingested for 12 days.

18. The method of claim 14, wherein said second nutritional supplement formulation further comprises one or more of Silybum marianum, Cynara scolymus, choline bitartrate, L-methionine, inositol, betaine HCl, lecithin, niacin, taraxacum, curcumin, alpha lipoic acid, folic acid, manganese, selenium, taurine, zinc, phyllanthus, picrorhiza, and trimethylglycine (TMG).

19. The method of claim 14, wherein said one or more sulfur-containing compounds in said third nutritional supplement formulation is one or more of L-glutathionine, N-acetyl-cysteine, dietary sulfur, selenomethionine, biotin, thiamine, and riboflavin.

20. The method of claim 19, wherein said third nutritional supplement formulation further comprises one or more of garlic, parsley, tumeric, neem, and triphala.

21. The method of claim 14, wherein said fourth nutritional supplement formulation further comprises one or more of bentonite powder, lactobacillus acidophilus, aloe vera powder, mint, anise, Vitamin C, bromelain, and magnesium.

22. The method of claim 15, wherein said nutritional supplement formulation comprises a time-release formulation of EDTA.

23. The method of claim 15, wherein said EDTA is included in said fifth nutritional supplement formulation at a dosage of about 500 to about 2500 mg/day.

24. The method of claim 14, further comprising ingesting a nutritional supplement formulation comprising one or more of EPA and GLA.

25. The method of claim 24, wherein said nutritional supplement provides a dose of about 3 g/day of said one or more of EPA and GLA.

26. A nutritional supplement formulation comprising Wasabia japonica.

27. The nutritional supplement formulation of claim 26, wherein said nutritional supplement formulation further comprises one or more of Silybum marianum, Cynara scolymus, choline bitartrate, L-methionine, inositol, betaine HCl, lecithin, niacin, taraxacum, curcumin, alpha lipoic acid, folic acid, manganese, selenium, taurine, zinc, phyllanthus, picrorhiza, and trimethylglycine (TMG).

28. The nutritional supplement formulation of claim 26, wherein said nutritional supplement formulation provides the following ingredients in the following ranges per day:

| | |
|---|---|
| a. Choline bitartrate | 750 mg-2000 mg |
| b. L-methionine | 5 mg-30 mg |
| c. Inositol | 50 mg-250 mg |
| d. Betaine HCl | 25 mg-150 mg |
| e. Lecithin | 25 mg-150 mg |
| f. Niacin | 2 mg-20 mg |
| g. Taraxacum | 10 mg-150 mg |
| h. Curcumin | 10 mg-150 mg |
| i. Silybum marianum | 10 mg-150 mg |
| j. Cynara | 10 mg-150 mg |
| k. Lipoic acid | 20 mg-150 mg |
| l. Folic acid | 50 μg-500 μg |
| m. Manganese | 1 mg-5 mg |
| n. Selenium | 25 μg-75 μg |
| o. Taurine | 2-8 g |
| p. Zinc | 2 mg-10 mg |
| q. Phyllanthus | 150 mg-2000 mg |
| r. Picrorhiza (kurroa) | 200 mg-800 mg |
| s. Wasabia japonica | 100 mg-2000 mg |
| t. Trimethylglycine | 150 mg-1500 mg. |

29. A nutritional supplement formulation comprising EDTA.

30. The nutritional supplement formulation of claim 29, wherein said nutritional supplement comprises a time-release formulation of EDTA.

31. The nutritional supplement formulation of claim 29, wherein said time-release formulation provides a dosage of EDTA of from about 500 to about 1500 mg.

32. A kit comprising the nutritional supplement formulation of claim 26.

33. The kit of claim 32, further comprising the nutritional supplement formulation of claim 28.

34. The kit of claim 32, further comprising a nutritional supplement formulation comprising one or more of psyllium powder, celery powder, prune concentrate, and flax seed powder in combination with one or more of betaine, pepsin, L-glutamine, FOS, and MSM.

35. The kit of claim 32, further comprising a nutritional supplement formulation comprising one or more of psyllium powder, celery powder, prune concentrate, and flax seed powder in combination with one or more of garlic, barley, and chlorella.

36. The kit of claim 32, further comprising a nutritional supplement formulation comprising one or more sulfur-containing compounds selected from the group consisting of L-glutathionine, N-acetyl-cysteine, dietary sulfur, selenomethionine, biotin, thiamine, and riboflavin.

37. A method of reducing inflammation in a human, the method comprising administering a therapeutically effective amount of a nutritional supplement formulation as defined in any one of claims 1, 14, 26, or 29 to a human.

38. The method of claim 37, wherein said reduction of inflammation is associated with a decreased level of C-reactive protein in the human as compared to the level of C-reactive protein in the human prior to administration of the nutritional supplement formulation.

39. The method of claim 37, wherein said reduction of inflammation can be correlated with a decline in a level of one or more inflammatory messengers.

40. The method of claim 39, wherein said inflammatory messenger is a leukotriene.

41. The method of claim 40, wherein said leukotriene is LTB₄.

42. The method of claim 39, wherein said inflammatory messenger is a prostaglandin.

43. The method of claim 42, wherein said prostaglandin is PGE₂.

44. The method of claim 39, wherein said reduction in a level of one or more inflammatory messengers is correlated with a decline in the level of arachadonic acid.

45. The method of claim 39, wherein said reduction in a level of one or more inflammatory messengers is correlated with an increase in the level of one or more of the following inflammatory messenger precursors: eicosapentaenoic acid, docosahexaenoic acid, gamma-linolenic acid, and dihomo gamma-linolenic acid.

46. A method of reducing a level of C-reactive protein in a human, the method comprising administering a therapeutically effective amount of a nutritional supplement formulation as defined in any one of claims 1, 14, 26, or 29 to a human.

47. A method of reducing a level of one or more inflammatory messengers in a human, the method comprising administering a therapeutically effective amount of a nutritional supplement formulation as defined in any one of claims 1, 14, 26, or 29 to a human.

48. The method of claim 47, wherein said inflammatory messenger is a leukotriene.

49. The method of claim 48, wherein said leukotriene is LTB₄.

50. The method of claim 47, wherein said inflammatory messenger is a prostaglandin.

51. The method of claim 50, wherein said prostaglandin is PGE₂.

52. A method of reducing a level of one or more inflammatory messenger precursors in a human, the method comprising administering a therapeutically effective amount of a nutritional supplement formulation as defined in any one of claims 1, 14, 26, or 29 to a human.

53. The method of claim 52, wherein said reduction in a level of one or more inflammatory messengers is correlated with a decline in the level of arachadonic acid.

54. The method of claim 52, wherein said reduction in a level of one or more inflammatory messengers is correlated with an increase in the level of one or more of the following inflammatory messenger precursors: eicosapentaenoic acid, docosahexaenoic acid, gamma-linolenic acid, and dihomo gamma-linolenic acid.

55. The method of claim 37, wherein said inflammation is acute inflammation.

56. The method of claim 37, wherein said inflammation is chronic inflammation.

57. The method of claim 37, wherein said human suffers from or is suspected of suffering from an autoimmune disease, an allergy, inflammatory bowel disease, obesity, asthma, gout, scleroderma, heart disease, rheumatoid arthritis, diabetes, Crohn's disease, and general aging.

58. A method of reducing a level of one or more heavy metals in a human, the method comprising administering a therapeutically effective amount of a nutritional supplement formulation as defined in any one of claims 1, 14, 26, or 29 to a human.

59. The method of claim 58, wherein said heavy metal is selected from mercury, aluminum, lead, cadmium, arsenic, and thallium.

60. The method of claim 59, wherein said heavy metal is mercury.

61. A method of modifying body composition in a human, the method comprising administering a therapeutically effective amount of a nutritional supplement formulation as defined in any one of claims 1, 14, 26, or 29 to a human.

62. The method of claim 61, wherein said modification comprises an increase in one or more of resting metabolic rate, fat free mass, and body weight.

63. The method of claim 61, wherein said modification comprises a decrease in one or more of body fat percentage, fat mass, and body weight.

64. The method of claim 61, wherein said modification in body composition is in comparison to the body composition measured in said human prior to the administration of the nutrition supplement formulation.
